(19) United States
(12) Patent Application Publication
(10) Pub. No.: US 2025/0269006 A1
(43) Pub. Date: Aug. 28, 2025

(54) METHODS FOR PREDICTING EFFICACY OF A MODIFIED LIVE PORCINE REPRODUCTIVE AND RESPIRATORY SYNDROME VIRUS (PRRSV) VACCINE

(71) Applicant: ELANCO US INC., Greenfield, IN (US)

(72) Inventors: Mark Hammer, Leland, NC (US); Jessica Proctor, Raleigh, NC (US); Tobias Kaeser, Raleigh, NC (US)

(21) Appl. No.: 18/866,887

(22) PCT Filed: May 12, 2023
(86) PCT No.: PCT/US2023/022074

§ 371 Date: Nov. 18, 2024

Related U.S. Application Data
(60) Provisional application No. 63/342,929, filed on May 17, 2022.

(51) Int. Cl. A61K 39/12; A61K 39/00; A61P 31/14; C12N 7/00; G01N 33/50; G01N 33/569; G01N 33/68

(52) U.S. Cl. CPC A61K 39/12; A61P 31/14; C12N 7/00; G01N 33/5091; G01N 33/56983; G01N 33/6854; G01N 33/6866; A61K 2039/552; A61K 2039/572; A61K 2039/575; C12N 2770/10034; G01N 2333/08; G01N 2333/57

(57) ABSTRACT

Methods are provided for eliciting heterologous immunogenicity against heterologous porcine reproductive and respiratory syndrome virus (PRRSV) strains to allow assessment of innate immunity and adaptive immunity. In other aspects are provided methods for determining the efficacy of a vaccine against PRRSV. In still other aspects are provided methods for predicting the efficacy of a vaccine against PRRSV in pigs suspected of having an infection with PRRSV.

### FIELD OF THE INVENTION

The present disclosure generally relates to methods for eliciting heterologous immunogenicity against heterologous porcine reproductive and respiratory syndrome virus (PRRSV) strains to allow assessment of innate immunity and adaptive immunity. In other aspects are provided methods for determining the efficacy of a vaccine against PRRSV. In still other aspects are provided methods for predicting the efficacy of a vaccine against PRRSV in pigs suspected of having an infection with PRRSV.

### BACKGROUND

The porcine reproductive and respiratory syndrome virus (PRRSV) continues to be the most economically important animal pathogen. This virus causes reproductive failure and respiratory disease, significantly contributing to the porcine respiratory disease complex (PRDC). See Lunney et al., “Porcine Reproductive and Respiratory Syndrome Virus (PRRSV): Pathogenesis and Interaction with the Immune System,” (2016) Ann. Rev Anim Biosci, 4: pp. 129-54. The respiratory diseases alone have caused an approximate 7.4% drop in annual production output, translating to over $664 million lost annually. See Valdes-Donoso et al., “Production Losses from an Endemic Animal Disease: Porcine Reproductive and Respiratory Syndrome (PRRS) in Selected Midwest US Sow Farms,” (2018) Front Vet Sci, 5, 102: doi:10.3389/fvets.2018.00102.

In addition to its immunosuppressive capacities, PRRSV's high mutation rate allows it to evade the hosts immunity provided either by infection or vaccination. See Loving et al., “Innate and adaptive immunity against Porcine Reproductive and Respiratory Syndrome Virus,” (2015) Vet Immunol Immunopathol 167: pp. 1-14; Geldhof et al., “Comparison of the efficacy of autogenous inactivated Porcine Reproductive and Respiratory Syndrome Virus (PRRSV) vaccines with that of commercial vaccines against homologous and heterologous challenges,” (2012) BMC Vet Res, 8, 182: doi:10.1186/1746-6148-8-182; Kvisgaard et al., “Challenge of Naive and Vaccinated Pigs with a Vaccine-Derived Recombinant Porcine Reproductive and Respiratory Syndrome Virus 1 Strain (Horsens Strain),” (2021) Vaccines (Basel), 9(5): doi:10.3390/vaccines9050417; Murtaugh et al., “Immunological solutions for treatment and prevention of porcine reproductive and respiratory syndrome (PRRS),” (2011) Vaccine, 29(46): pp. 8192-204; Rowland et al., “Alternative strategies for the control and elimination of PRRS,” (2017) Vet Microbiol 209: pp. 1-4; Shi et al., “Molecular epidemiology of PRRSV: a phylogenetic perspective,” (2010) Virus Res 154(1-2): pp. 7-17; Zhou et al., “Efficacy evaluation of three modified-live virus vaccines against a strain of porcine reproductive and respiratory syndrome virus NADC30-like,” (2017) Vet Microbiol 207: pp. 108-116.

These mutations led to a plethora of strains: PRRSV can be divided into two species, type-1 or PRRSV-1, mainly found in Europe, and type-2 or PRRSV-2, prevalent in North America. Based on the open reading frame (ORF), PRRSV-2 is further divided into nine lineages with numerous PRRSV strains. This high diversity leads to a strong challenge for PRRSV vaccines: they need to protect against the various constantly evolving PRRSV strains present in the swine industry. The most prevalent PRRSV-2 lineages currently are lineage 1, 5, 8, and 9. See Brar et al., “Genomic evolution of porcine reproductive and respiratory syndrome virus (PRRSV) isolates revealed by deep sequencing,” (2014) PLoS One 9(4): e88807, doi:10.1371/joumal.pone.0088807.

However, based on the high prevalence of these strains, what is needed is a PRRSV vaccine that the industry is confident can provide broad cross-reactivity against the various PRRSV strains.

The current disclosure addresses this need.

### SUMMARY OF THE DISCLOSURE

The inventors have discovered methods for eliciting heterologous immunogenicity against heterologous porcine reproductive and respiratory syndrome virus (PRRSV) strains to allow assessment of innate immunity and adaptive immunity. In other aspects are provided methods for determining the efficacy of a vaccine against PRRSV. In still other aspects are provided methods for predicting the efficacy of a vaccine against PRRSV in pigs suspected of having an infection with PRRSV.

In one embodiment is provided a method for eliciting heterologous immunogenicity against heterologous porcine reproductive and respiratory syndrome virus (PRRSV) strains to allow assessment of innate immunity and adaptive immunity. The method comprises first administering to a pig of an effective amount of a modified live PRRSV vaccine or a control injection. At about 28 days post-vaccine administration, the pig is challenged the pig with an intranasal inoculation of an amount of a live PRRSV of a known strain. In the pig are measured temperature and weight immediately prior to administration of the modified live PRRSV vaccine, immediately prior to the challenge with the intranasal inoculation of the known PRRSV strain, and at least 7- and 14-days post challenge. Blood samples are obtained from the pig immediately prior to administration of the modified live PRRSV vaccine, immediately prior to the challenge with the intranasal inoculation of the known PRSSV strain, and at least 7- and 14-days post challenge. Various immune correlates of protection (CoP) are then measured and determined from each blood sample. Measurements include a CD4 T-cell response, the presence of strain-specific neutralizing antibodies, the presence of CD4, CD8, and TCR-γδ cells, IFN-γ levels, and the amount of PRRSV-specific immunoglobulin A (IgA) and immunoglobulin G (IgG) levels. All measurements from vaccinated pigs are compared to those obtained from control injected pigs.

In any of the embodiments, the known porcine reproductive and respiratory syndrome virus (PRRSV) strain is selected from PRRSV type-1 (PRRSV-1) and PRRSV type-2 (PRRSV-2) virus strain. In other embodiments, the known porcine reproductive and respiratory syndrome virus (PRRSV) strain is the PRRSV type-2 (PRRSV-2) virus strain selected from the group consisting of NADC30 and NC174 (lineage 1), VR2332 (lineage 5), and NADC20 (lineage 8).

In any of the embodiments, the strain-specific neutralizing antibodies are one or more of anti-NADC30, anti-VR2332, and anti-NADC20 neutralizing antibodies.

In yet other embodiments, the administering to a pig of an effective amount of a modified live PRRSV vaccine followed by the challenging the pig with an intranasal inoculation of an amount of a known live PRRSV strain at least 28 days post-vaccine administration induces immune correlates of protection (CoP) marked by: i) an increase in T-cell activation as evidenced by a differentiation of CD4 T and CD8 cells; ii) an increase in the amount of PRRSV-specific immunoglobulin G (IgG) levels; iii) the production of serum neutralizing antibodies; and, iv) an increase of serum IFN-γ levels, as compared to control injections.

In any of the embodiments, the methods include the isolation, storage and banking of peripheral blood mononuclear cells (PBMC) obtained from the blood samples from pigs administered an effective amount of a modified live PRRSV vaccine.

Another embodiment of the invention provides a method for determining the efficacy of a vaccine against porcine reproductive and respiratory syndrome virus (PRRSV) comprising: i) administering to a pig of an effective amount of a modified live PRRSV vaccine; ii) challenging the pig with an intranasal inoculation of an amount of a live PRSSV known strain at least 28 days post-vaccine administration; iii) measuring in the pig, temperature, and weight immediately prior to administration of the modified live PRRSV vaccine, immediately prior to the challenge with the intranasal inoculation of the known PRRSV strain, and at least 7- and 14-days post challenge; iv) obtaining blood samples, nasal swabs from the pig immediately prior to administration of the modified live PRRSV vaccine, immediately prior to the challenge with the intranasal inoculation of the known PRRSV strain, and at least 7- and 14-days post challenge; v) assessing lung and lymph node pathology in the pig upon necropsy; vi) obtaining a bronchoaveolar lavage samples upon necropsy; vii) measuring in each blood sample, nasal swab, and bronchoaveolar lavage sample, the amount of virus present, the amount of PRRSV-specific immunoglobulin A and immunoglobulin G; and, viii) comparison of all measurements with those obtained from control injected pigs.

In some of the embodiments, where the administering to a pig of an effective amount of a modified live PRRSV vaccine followed by the challenging the pig with an intranasal inoculation of an amount of a known live PRRSV strain at least 28 days post-vaccine administration can induce: i) little or no lung and lymph node pathology in the pig upon necropsy; ii) a decrease in the amount of PRRSV virus in samples obtained from blood, nasal swabs and bronchoaveolar lavage upon necropsy; and, iii) an increase in the amount of PRRSV-specific immunoglobulin A and immunoglobulin G; as compared to measurements with those obtained from control injected pigs.

In yet another embodiment of the invention is provided a method of predicting the efficacy of a vaccine against porcine reproductive and respiratory syndrome virus (PRRSV) a pig suspected of having an infection with PRRSV, comprising: i) isolating PRRSV from a blood or nasal swab sample obtained from the pig suspected of having an infection with PRRSV; ii) challenging with the PRRSV from the pig suspected of having an infection with PRRSV, isolated, stored and banked samples of peripheral blood mononuclear cells (PBMC) previously obtained from the blood samples from pigs administered an effective amount of a modified live PRRSV vaccine wherein the pigs were further challenged with an intranasal inoculation of an amount of a live PRRSV known strain at least 28 days post-vaccine administration and their PBMC's isolated, stored and banked wherein the CD4 T and CD8 T-cell response as the differentiation of CD4 T and CD8 cells was previously obtained; iii) measuring the immune correlate of protection (CoP) as the CD4 T and CD8 T-cell response in the PBMC's following the challenge with the PRRSV from the pig suspected of having an infection with PRRSV; and iv) comparing the CD4 T and CD8 T-cell response of step iii) with the previously obtained CD4 T and CD8 T-cell response from the isolated, stored and banked PBMC samples of step ii).

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

### BRIEF DESCRIPTION OF THE DRAWINGS

FIG. 1 depicts the PRRSV type-2 challenge in an in vivo animal trial layout. Four-week-old weaner pigs were distributed into ten groups-five MOCK (i.e. phosphate buffered saline)- and five Prevacent®-vaccinated groups. Pigs were vaccinated at −28 days post challenge (dpc). At day of challenge (0 dpc), each of the five groups received one of five intranasal inoculations—MOCK, NADC30, NC174, VR2332, or NADC20. At 14 dpc, pigs were sacrificed to assess lung pathology. As indicated in the timeline, blood and nasal swabs were collected throughout the study to assess viral loads as well as the humoral and T-cell immune response.

FIG. 2A-C depicts the heterologous vaccine efficacy of Prevacent®. Rectal temperatures (FIG. 2A), viremia (FIG. 2B), and (FIG. 2C) viral loads in nasal swabs were determined at 0, 7, and 14 days post challenge (dpc) with MOCK (grey), or the PRRSV strains 1-4-4 (NADC30, dark blue), NC174 (red), VR2332 (green), or 1-4-2 (NADC20, light blue). The line graphs in (FIG. 2A) illustrate the means with standard deviation of rectal temperatures [° C.]. Viremia (FIG. 2B) and viral shedding (FIG. 2C) were quantified by PRRSV-specific RT-qPCR in serum and nasal swabs, respectively (genomic copy numbers/mL [log 10]). The black bars represent the median values; in addition, individual data points are shown for MOCK vaccinated animals (open diamonds) and MLV vaccinated animals (filled squares). The data were analyzed using Sidik multiple comparison test 2-way ANOVA. Each vaccinated group was compared to their respective PRRSV type-2 challenge MOCK vaccinated group within each timepoint. ****p<0.0001. ***p<0.001, **p<0.01, *p<0.05.

FIG. 3A-C depicts the heterologous vaccine efficacy on lung viral loads, pathology, and inguinal lymph nodes size. (FIG. 3A) Viral loads in lung were assessed in bronchoalveolar lavage (BAL) by PRRSV-specific RT-qPCR (genomic copy numbers/mL [log 10]). Of note, while PRRSV was not detected in BAL of one MOCK-vaccinated/NADC20-challenged animal, this pig had the second highest lung pathology score combined with the highest PRRSV load in the 14 dpc nasal swab. Based on this discrepancy, it was excluded from this analysis. (FIGS. 3B and C) Lung gross- and histopathology of all seven lobes were assessed by a blinded veterinarian at 14 days post challenge (dpc). (FIG. 3B) depicts the percental lung lesions for each individual pig. (FIG. 3C) shows the histopathology scores of all seven lobes following the scoring guidelines of Halbur et al. See Halbur et al. “Comparison of the pathogenicity of two US porcine reproductive and respiratory syndrome virus isolates with that of the Lelystad virus,” (1995) Vet Pathol. 32(6): pp. 648-60. Each vaccinated group was compared to their respected PRRSV type-2 challenge unvaccinated group using a two-tailed unpaired t-test. The black bars represent the median values; in addition, individual data points are shown for MOCK vaccinated animals (open diamonds) and MLV vaccinated animals (filled squares). Each vaccinated group was compared to their respective PRRSV type-2 challenge unvaccinated group. Data comparison was performed using a two-tailed unpaired t-test. ****p<0.0001. ***p<0.001, **p<0.01, *p<0.05.

FIG. 4A-D depicts the heterologous vaccine immunogenicity as the humoral immune response. Immunoglobulin A of (FIG. 4A) bronchoalveolar lavage (BAL), (FIG. 4B) nasal swabs, and (FIG. 4C) serum IgG levels were evaluated at 0 and 14 days post challenge (dpc) via a PRRSV×3 ELISA. The IgA and IgG ELISA S/P ratios were compared within their challenge groups—MOCK (grey), 1-4-4 (NADC30, dark blue), NC174 (red), VR2332 (green), and 1-4-2 (NADC20, light blue). The black bars represent the median values; in addition, individual data points are shown for MOCK vaccinated animals (open diamonds) and MLV vaccinated animals (filled squares). Data were statistically analyzed using a 2-way ANOVA with time and vaccination as the two parameters and Tukey's multiple comparison test. ****p<0.0001. ***p<0.001, **p<0.01, *p<0.05. (FIG. 4D) Neutralizing antibody (NA) titers determined via FFN test against the respective challenge strain at 0 and 14 dpc. Since no animals showed FFN titers at 0 dpc, only the 14 dpc data are shown. Titers≥1:4 were considered positive. The titer for each individual PRRSV-challenged pig is shown. Positive NA titers are highlighted in blue (NADC30), red (NC174, not detected), green (VR2332), and light blue (NADC20).

FIG. 5A-D shows the heterologous vaccine immunogenicity as the proliferation of CD4, CD8, and TCR-γδ T cells. (FIG. 5A) shows the gating hierarchy to assess the heterologous proliferative response of T-cell subsets to the respective PRRSV type-2 challenge strains. A live/dead discrimination dye was included to exclude dead cells. Live cells were used to identify live lymphocytes via a FSC/SSC lymphocyte gate. From live lymphocytes, doublets were excluded using a FSC-width (FSC-W)/FSC-area (FSC-A) gate on singlets. These single living lymphocytes were used to gate on T cells (FSC-A/CD3), and further to discriminate TCR-αβ and TCR-γδ T cells. TCR-αβ T cells were further divided into CD4 and CD8 T cells via their CD4/CD8α expression profile. Proliferation of the CD4, CD8, and TCR-γδ T cells was identified via a violet proliferation dye. Two examples demonstrate representative staining patterns of a control animal (top right plot) and a high responder animal (bottom right plot). (FIG. 5B-D) show the proliferative responses of CD4 (FIG. 5B), CD8 (FIG. 5C), and TCR-γδ T cells (FIG. 5D) according to their challenge groups—MOCK (grey), NC174 (red), NADC20 (light blue), NADC30 (dark blue) and VR2332 (green). The black bars represent the median values; in addition, individual data points are shown for MOCK vaccinated animals (open diamonds) and MLV vaccinated animals (filled squares). Data were statistically analyzed using a 2-way ANOVA with time and vaccination as the two parameters and Tukey's multiple comparison test. ****p<0.0001. ***p<0.001, **p<0.01, *p<0.05.

FIG. 6A-D show the heterologous vaccine immunogenicity as IFN-γ production of CD4, CD8, and TCR-γδ T cells. (FIG. 6A) Gating hierarchy to assess the heterologous IFN-γ response of T-cell subsets to the respective PRRSV type-2 challenge strains. The gating hierarchy follows largely the proliferation analysis shown in FIG. 5. However, instead of gating on proliferating cells, IFN-γ was analyzed in an FSC-A/IFN-γ plot. The IFN-γ gate was set using the appropriate FMO control (top right plot). (FIG. 6B-D) show the IFN-γ responses of CD4 (FIG. 6B), CD8 (FIG. 6C) and TCR-γδ T cells (FIG. 6D) according to their challenge groups—MOCK (grey), 1-4-4 (NADC30, dark blue), NC174 (red), VR2332 (green), and 1-4-2 (NADC20, light blue). The black bars represent the median values; in addition, individual data points are shown for MOCK vaccinated animals (open diamonds) and MLV vaccinated animals (filled squares). Data were statistically analyzed using a 2-way ANOVA with time and vaccination as the two parameters and Tukey's multiple comparison test. ****p<0.0001. ***p<0.001, **p<0.01, *p<0.05.

FIG. 7A-B depict the heterologous vaccine immunogenicity as the differentiation of IFN-γ producing CD4 T cells. (FIG. 7A) shows the gating hierarchy to assess the differentiation of IFN-γ producing CD4 T cells. After gating on IFN-γ+CD4 T cells as described in FIG. 6, their differentiation was analyzed via their CD4/CD8a expression profile to distinguish naïve (CCR7+CD8α−), central memory (T_{CM} CCR7+CD8α+) and effector memory (T_{EM}, CCR7-CD8α+) CD4 T cells (top right plot). Since the vast majority of CD8α+IFN-γ-producing CD4 T cells belonged to the T_{CM} subset (data not shown), both T_{CM} and T_{EM} were combined in the downstream analysis into the “memory/effector” subset. (FIG. 7B) shows the frequency of these memory/effector within IFN-γ-producing CD4 T cells according to their challenge groups—MOCK (grey), 1-4-4 (NADC30, dark blue), NC174 (red), VR2332 (green), and 1-4-2 (NADC20, light blue). The black bars represent the median values; in addition, individual data points are shown for MOCK vaccinated animals (open diamonds) and MLV vaccinated animals (filled squares). Data were statistically analyzed using a 2-way ANOVA with time and vaccination as the two parameters and Tukey's multiple comparison test. ****p<0.0001. ***p<0.001, **p<0.01, *p<0.05.

FIG. 8 shows the quantification of the Prevacent®k vaccine strain in serum. The prevalence of the Prevacent® vaccine strain was quantified via Prevacent®-specific RT-qPCR. This table shows the Ct values of MOCK, NADC20, NC174, VR2332, and NADC20 challenged animals at 7 days post challenge.

### DETAILED DESCRIPTION

Throughout this disclosure, various quantities, such as amounts, sizes, dimensions, proportions, and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The term “subject” or “patient” as used herein, refers to a mammal, more particularly a pig or swine.

The embodiments and features of the disclosure will become more apparent through reference to the following description, the accompanying figures, and the claims. Furthermore, it is to be understood that the features of the various embodiments described herein are not mutually exclusive and can exist in various combinations and permutations.

The present disclosure is based on the knowledge that porcine reproductive and respiratory syndrome virus (PRRSV) vaccines, although available in North America for almost 30 years, face a significant hurdle: they must provide cross-protection against the highly diverse PRRSV strains. This cross-protection, or heterologous vaccine efficacy, relies greatly on the vaccine's ability to induce a strong immune response against various strains—heterologous immunogenicity.

The inventors investigated vaccine efficacy and immunogenicity of a modified live virus (MLV) against four heterologous type 2 PRRSV (PRRSV-2) strains as a model for testing the vaccine efficacy and immunogenicity of a modified live virus (MLV) against any suspected strain of PRRSV. In the current disclosure, pigs were divided into several groups. Half were MOCK-vaccinated (i.e. with phosphate-buffered saline), and the other half vaccinated with the the modifiled live PRSSV vaccine, Prevacent® PRRS MLV vaccine (Elanco, Inc.). Four weeks after vaccination, groups were challenged with either MOCK, or four PRRSV-2 strains—NC174 or NADC30 (both lineage 1), VR2332 (lineage 5), or NADC20 (lineage 8). Pre- and post-challenge, lung pathology, viral loads in both nasal swabs and sera, anti-PRRSV IgA/G, neutralizing antibodies, and the PRRSV-2 strain-specific T-cell response were evaluated. At necropsy, lungs were harvested to assess viral loads, pathology, and IgA levels in BAL. Lung pathology was only induced by NC174, NADC20, and NADC30; within these, vaccination did reduce pathology of the NADC20 and NADC30 strains. All pigs became viremic and vaccinated pigs had decreased viremia upon challenge with NADC20, NADC30 and VR2332. Regarding vaccine immunogenicity, vaccination induced a strong systemic IgG response and boosted the post-challenge serum IgG levels for all strains. Furthermore, vaccination increased the number of animals with neutralizing antibodies against three of the four challenge strains—NADC20, NADC30, and VR2332. The heterologous T-cell response was also improved by vaccination: not only did vaccination increase the induction of heterologous effector/memory CD4 T cells, but it also improved the heterologous CD4 and CD8 proliferative and/or IFN-γ response against all strains. Importantly, correlation analyses revealed that the (non-PRRSV strain-specific) serum IgG levels and the PRRSV strain-specific CD4 T-cell response were the best immune correlates of protection. Overall, the Prevacent® elicited various degrees of efficacy and immunogenicity against different heterologous strains from three diverse PRRSV-2 lineages to serve as a model as for testing the vaccine efficacy and immunogenicity of a modified live virus (MLV) against any suspected strain of PRRSV.

### Vaccine Imnunogenicity

One aspect of the present disclosure includes an analysis and confirmation of vaccine immunogenicity analysis included both the humoral and T-cell immune response following immunization with a modified live PRRSV vaccine in order develop methods and test for determining the efficacy of modified live PRRSV vaccines against unknow or suspected strains of PRRSV virus strains.

Neutralizing antibodies (nAbs) play a critical role in both viral clearance and defense against re-infection. See Lopez et al., “Role of neutralizing antibodies in PRRSV protective immunity,” (2004) Vet Immunol Immunopathol 102(3): pp. 155-163. However, several studies have noted the postponed induction of nAbs prior to clearance of viremia. Seefor example Lunney et al. 2016; Butler et al., “Antibody Repertoire Development in Swine, (2017) Ann. Rev Anim Biosci 5: pp. 255-279; Pileri et al. “Review on the transmission porcine reproductive and respiratory syndrome virus between pigs and farms and impact on vaccination,” (2016) Vet Res 47(1): p. 108. These current studies emphasized that clearance of PRRSV viremia can occur before the presence of nAbs.

Therefore, while developing nAbs are important in the protection against PRRSV, other factors seem to play a relevant role as well. Viremia reduction or even viral clearance in the absence of nAbs is partly explained by the cell-mediated immune response including the T-cell response, such as IFN-γ production by CD4, CD8, and TCR-γδ T cells. See Chae et al., “Commercial PRRS Modified-Live Virus Vaccines,” (2021) Vaccines (Basel) 9(2): doi:10.3390/vaccines9020185; Kick et al., “The T-Cell Response to Type 2 Porcine Reproductive and Respiratory Syndrome Virus (PRRSV), (2019) Viruses, 11(9): doi:10.3390/v11090796; Nan et al., “Improved Vaccine against PRRSV: Current Progress and Future Perspective,” (2017) Front Microbiol 8: pp. 1635. Based on this central role of T cells in the control of PRRSV, the inventors provide herein a detailed analysis of the PRRSV-strain specific proliferative and IFN-γ response of CD4, CD8, and TCR-γδ T cells. Responding CD4 T cells were additionally analyzed on their differentiation from CD8α-naïve into CD8α+ antigen-experienced memory/effector cells. This differentiation allows the distinction between a primary and a secondary response of these CD4 T cells.

### Design Summary

To accomplish the study of that detailed heterologous vaccine immunogenicity and efficacy, sixty pigs were distributed into ten groups—five immunized with the commercially available modified live PRRSV vaccine, Prevacent® PRRS MLV vaccine (Elanco, Greenfield, IN) and five MOCK-inoculated (phosphate-buffered saline). Four weeks post vaccination, pigs were challenged with one of the above-mentioned PRRSV-2 strains or MOCK-inoculated. Viral shedding and viremia as well as the induced immune response were followed for two weeks; then, pigs were sacrificed to additionally assess viral loads in bronchoalveolar lavage (BAL), lung gross- and histopathology, and inguinal lymph node size. The immune response analysis included the humoral and T-cell immune response: the humoral response was studied not only by quantifying mucosal IgA in nasal swabs and bronchoalveolar lavage (BAL), but also by determining the serum IgG and nAb levels; the systemic T-cell response was analyzed in detail including the proliferative and IFN-γ response of CD4, CD8, and TCR-γδ T cells as well as CD4 T-cell differentiation. In addition, these immune parameters were investigated for their correlation, i.e. immune correlates to the studied vaccine efficacy parameters—lung pathology, viral shedding, and viremia.

It was discovered that Prevacent® induced various levels of heterologous immunity, including inducing a strong IgA response in the BAL, a strong systemic IgG response, and increasing the prevalence of anti-NADC30, -VR2332, and -NADC20 nAbs. Further, immunization with Prevacent® also promoted i) the CD4 T-cell differentiation, ii) the proliferation of CD4, CD8, and TCR-γδ cells, and iii) a stronger post-challenge IFN-γ response.

The correlation analyses between the vaccine efficacy and immunogenicity parameters revealed two important immune correlates of protection—systemic IgG levels and the CD4 T-cell response. However, in contrast to the ELISA used to quantify the systemic IgG levels, the in vitro restimulation method followed by multi-color flow cytometry, determines the CD4 response specific to the challenge strain. Conclusively, for the first time, the inventors identified the systemic CD4 T-cell response as a strain-specific immune correlate of protection (CoP) for PRRSV-2.

Thus, the inventors discovered that the immune correlates of protection (CoP) can strongly facilitate vaccine development as well as being used to predict vaccine efficacy against newly emerging PRRSV-2 strains.

### Study Design

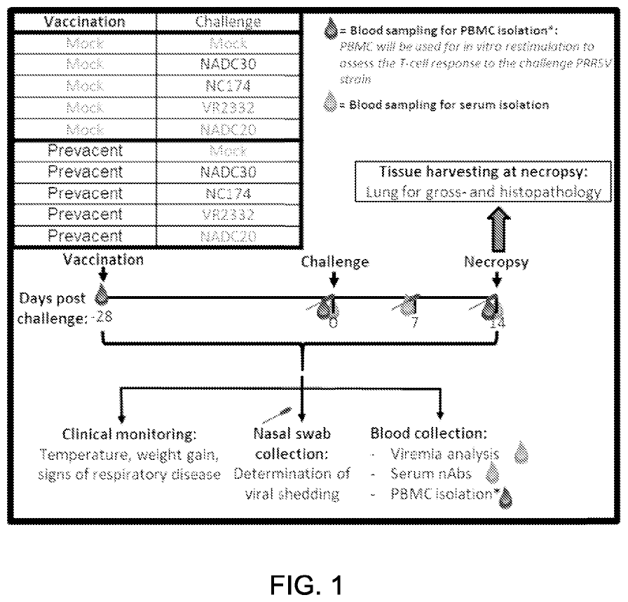

The study design of the present disclosure is illustrated in FIG. 1. Sixty 4-week-old weaners from a PRRSV-2-negative farm (NC State University Swine Education Unit, Raleigh, NC, USA) were brought to a BSL-2 Laboratory Animal Research—LAR facility at NC State University, College of Veterinary Medicine (Raleigh, NC, USA). These 60 weaners were randomly divided into ten groups using a GraphPad online randomization tool. Five groups were intramuscularly (IM) MOCK-inoculated with phosphate-buffered saline (PBS), and five groups with Prevacent® as recommended by the manufacturer. Twenty-eight days after vaccination, pigs were intranasally challenged using a Nasal Mist Intranasal Mucosal Atomization Device (Mountainside Medical Equipment, Marcy, NY) (500 L/nostril; lmL total).

Each of the MOCK and MLV vaccinated groups was challenged with a 106 TCID50/mL dose of either NC174 (lineage 1A), NADC30 (lineage 1C), VR2332 (lineage 5), or NADC20 (lineage 8). MOCK challenged pigs were challenged with either 1% bovine serum albumin (BSA) in PBS (3/6 pigs) or Opti-MEM™ (3/6 pigs) as these were the two-suspension media used for the different viral strains. Pigs were clinically monitored daily. At −28-, 0-, 7-, and 14-days post-challenge (dpc), blood was collected for serum and/or isolation of peripheral blood mononuclear cells (PBMC). Body weight and rectal temperatures were recorded weekly. To facilitate the handling, necropsy was performed over two days −15 and 16 dpc. Pigs were euthanized using lethal injection and lungs were harvested.

First, lungs were assessed for gross pathology and photographs taken for documentation. Then, lungs were filled with 50 mL PBS, gently massaged and bronchoalveolar lavage (BAL) harvested for downstream assessment of lung viral loads and the local humoral and cellular immune response. Thereafter, tissue samples were taken for histopathology quantification and the characterization of the infiltrated lung tissue T cells. Inguinal lymph nodes were also harvested and weighted as clinical indicator ofPRRSV exposure. See Rossow et al., “Pathogenesis of porcine reproductive and respiratory syndrome virus infection in gnotobiotic pigs,” (1995) Vet Pathol 32(4): pp. 361-73. The experimental procedures were approved by the NC State University Institutional Animal Care and Use Committee (IACUC) ID #17-166A (Nov. 29, 2017).

### PRRSV Strains

NC174, NADC20 andNADC30 were provided by Elanco. VR2332 was produced and titrated in house on MA-104 cells. Serum pools from MOCK-vaccinated, challenged pigs at 7 dpc were sent to ISU VDL for ORF5 sequencing: the sequence analysis confirmed the correct identity of the challenge strains (data not shown, d.n.s).

### Processing of Bronchoalveolar Lavage, Nasal Swabs and Blood

Aliquots of 0.4 mL BAL were added to 0.6 mL TriReagent (Ambion, Austin, TX, USA), mixed and stored at −80° C. for downstream PRRSV quantification via qPCR. Remaining BAL was centrifuged at 400 g and 4° C. for ten minutes to pellet BAL immune cells. Cell pellets were harvested, counted, and used for the analysis of the local T-cell immune response. Supernatants were aliquoted and stored at −80° C. for downstream analysis of the humoral immune response via IgA ELISA. Nasal swabs were rotated in each nostril and placed in tubes filled with 1 mL PBS. After collection, swabs were vortexed and then rotated in a circular motion pressing against the tube wall before removal of the swabs from the tube. The PBS from these nasal swabs was aliquoted and stored at −80° C. for downstream PRRSV and antibody quantification. Whole blood for serum isolation was collected in SST tubes (BD Bioscience, San Jose, CA, USA) and incubated upright for 30 minutes. After incubation, blood was spun at 2,000 g for 20 mins at 23° C. Serum was harvested and stored in aliquots at −80° C. Whole blood for peripheral blood mononuclear cell (PBMC) isolation was collected in Heparin tubes (BD Bioscience). Isolation of PBMC was performed by density centrifugation using Sepmate tubes (StemCell, Vancouver, Canada) and Ficoll-Paque (GE Healthcare, Uppsala, Sweden). After isolation, PBMCs were used fresh for in vitro restimulation to study the PRRSV-strain specific T-cell immune response.

### Viremia and Viral Loads

Isolated serum and nasal swabs were shipped to Iowa State University Veterinary Diagnostic Laboratory (ISU VDL) (Ames, IA, USA) for PRRSV quantification using either a PRRSV-universal or an “Elanco Prevacent®-like” specific reverse transcription (RT) quantitative PCR. Results were given as Ct values (“Elanco Prevacent®-like” RT-qPCR) or genomic copy numbers/mL (universal RT-qPCR).

### Serum Anti-PRRSV IgG and Anti-PRRSV IgA

Isolated serum and nasal swabs were shipped to ISU VDL. Serum IgG levels were determined with PRRSV×3 enzyme-linked immunosorbent assay (ELISA, IDEXX, Westbrook, ME, USA). PRRSV Oral fluid IgA ELISA was used to determine IgA of nasal swabs.

### Neutralizing Antibodies

Serum samples at 0 and 14 dpc were shipped to South Dakota State University Animal Research and Diagnostic Laboratory (SDSU ARDL). Neutralizing antibodies were measured by the fluorescent focus neutralization (FFN) test. See Valdes-Donoso et al. (2018). A titer of >1:4 was considered positive. Isolated serum was tested against the respective homologous challenge strain. Both MOCK-challenged groups were tested against all four viral strains.

### Lung Gross Pathology, Histology, and Lymph Node Weight

At necropsy, lungs and inguinal lymph nodes were harvested. Photos of the dorsal and ventral sides of the lungs were taken. Lobes were scored by a blinded veterinarian. For histopathology assessment, tissue from seven lung lobes were extracted—left apical, left cardiac, left diaphragmatic (caudal), right apical, right cardiac, right diaphragmatic (caudal), and intermediate (accessory). Tissue samples were fixed in Formaldehyde/Zn fixative (Electron Microscopy Sciences, Hatfield, PA) for twenty-four hours; then, they were transferred to 70% ethanol. The tissue processing, hematoxylin and eosin (H&E) staining, and slide preparation were performed by the NC State University Histology lab. Histopathology was assessed by a blinded pathologist as previously described by Halbur et al. (1995). Briefly, scores were recorded as (0) normal, (1) slightly altered, (2) mild, (3) moderate, or (4) severe. For a general assessment of immune activation, both inguinal lymph nodes were collected at sacrifice and weighed in grams.

### The PRRSV Challenge-Strain Specific Proliferation of T Cells

To measure the proliferation of PRRSV-specific T-cell subsets, freshly isolated PBMCs were stained with CellTrace™ Violet cell Proliferation Kit (Invitrogen) according to the manufacturer's instructions. Stained cells were seeded in 96-well round-bottom plates (Sarstedt, Nümbrecht, Germany) at 200,000 cells/well. Cells were stimulated for 72 hours with medium (MOCK), NC174, NADC20, NADC30, or VR2332 (MOI of 0.1); Concanavalin A (ConA, 2.5 μg/mL, Alfa Aesar) stimulation as a positive control. Cells from eight replicates were pooled and stained for flow cytometry analysis according to Table 1. Flow cytometry data were acquired on a Cytoflex using the CytExpert software (Beckman Coulter). Data analysis was performed with FlowJo version 10.5.3 (FLOWJO LLC) with gates based upon relevant FMO controls.

TABLE 1
Flow Cytometry Staining Panel

| | | | | Labeling | Primary Ab | 2^{nd} Ab |
|---|---|---|---|---|---|---|
| Antigen | Clone | Isotype | Fluorochrome | Strategy | source | Source |
| CD3 | PPT3 | IgG1 | FITC | Directly | Southern | — |
| | | | | conjugated | Biotech | |
| CD4 | 74-12-4 | IgG2b | Brilliant | Secondary | BEI | Jackson |
| | | | Violet 480 | antibody | Resources | Immunoresearch |
| CD8α | 76-2-11 | IgG2a | Brilliant | Biotin- | Southern | Biolegend |
| | | | Violet 605 | Streptavidin | Biotech | |
| TCR-γδ | PGBL22A | IgG1 | Alexa | Kingfisher | Invirtogen | — |
| | | | Flour 647 | | | |
| CCR7 | 3D12 | rIgG2a | Brilliant | Directly | BD | — |
| | | | Blue 700 | Conjugated | Biosciences | |
| Live/Dead | — | — | Near | — | Invitrogen | — |
| | | | Infrared | | | |
| IFN-γ* | P2G10 | IgG1 | PE | Directly | BD | — |
| | | | | conjugated | Biosciences | |
| Proliferation^{#} | — | — | CellTrace ™ | — | Invitrogen | — |
| | | | Violet | | | |

While the CD3, CD4, CD8α, TCR-γδ, CRR7, and Live/Dead staining was included in both panels, the IFN-γ staining (*) was only included in the IFN-γ analysis and the proliferation (^{#}) staining only in the proliferation analysis.

### The PRRSV Challenge-Strain Specific IFN-γ Production of T Cells

PBMCs were plated at 500,000 cells/well and allowed to rest overnight. The following day, cells were stimulated in with either media (MOCK), NC174, NADC20, NADC30, or VR2332 MOI of 0.1); Phorbol 12-myristate 13-acetate (PMA, 5 ng/mL, Alfa Aesar, Ward Hill, MA, USA)/Ionomycin (500 ng/mL, AdipoGen, San Diego, CA, USA) was used as a positive control. Plates were cultured for 18 hrs; Monensin (5 μg/mL, Alfa Aesar) was added for the last 4 hrs of culture. Eight replicates were then pooled and stained for flow cytometry analysis according to Table 1. Data were acquired on a Cytoflex using the CytExpert software (Beckman Coulter). Data analysis was performed with FlowJo version 10.5.3 with gates based upon the FMO controls.

### Statistical Analysis

Statistical analysis was performed using GraphPad Prism 9.1.1 (GraphPad Software, San Diego, CA). All qPCR data were log-transformed prior to statistical analysis. Depending on the dataset, statistical significance was analyzed by either two-way ANOVA or a two-tailed unpaired Student t-test. Multiple comparisons were performed using either Tukey's or Šidák multiple comparisons test.

Further reference is made to the following experimental examples which are given for the purpose of illustrating various embodiments of the invention and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art.

### EXAMPLES

### Example 1

### Heterologous Vaccine Efficacy

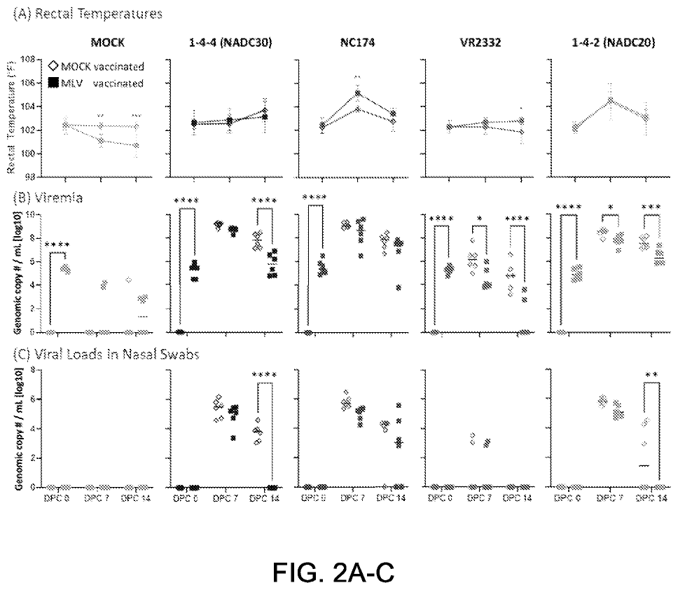

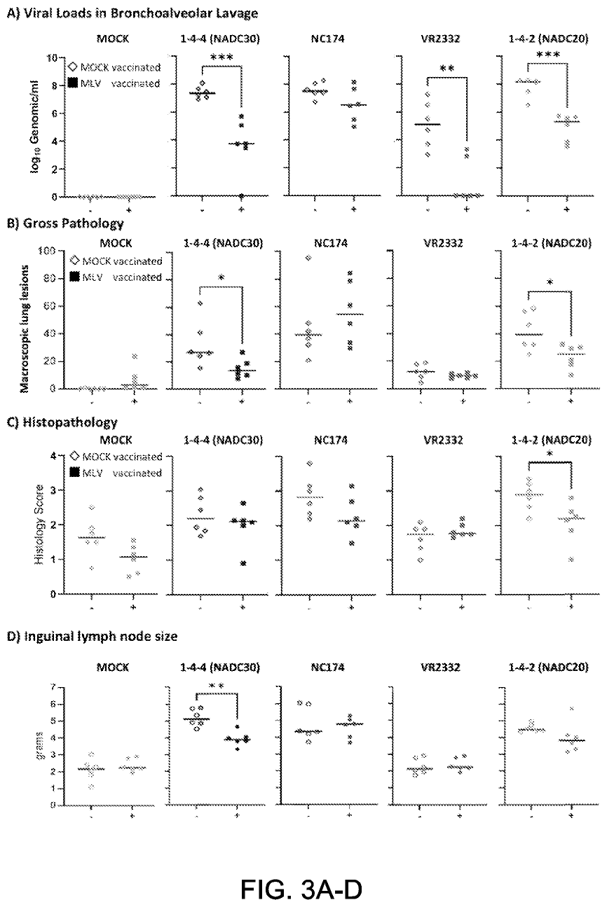

The heterologous vaccine efficacy of Prevacent® was determined in three ways: i) clinical signs including rectal temperatures, ii) PRRSV loads in nasal swabs and serum were assessed at 0, 7, and 14 dpc (FIG. 2); and iii) viral loads, and the lung gross and histopathology were assessed at necropsy (14 dpc, FIG. 3).

### Weight Gains, Clinical Signs and Viral Loads in Serum, BAL, and Nasal Swabs

FIG. 2A-C depicts the heterologous vaccine efficacy of Prevacent®. Rectal temperatures (FIG. 2A), viremia (FIG. 2B), and (FIG. 2C) viral loads in nasal swabs were determined at 0, 7, and 14 days post challenge (dpc) with MOCK (grey), or the PRRSV strains 1-4-4 (NADC30, dark blue), NC174 (red), VR2332 (green), or 1-4-2 (NADC20, light blue). The line graphs in (FIG. 2A) illustrate the means with standard deviation of rectal temperatures [° C.]. Viremia (FIG. 2B) and viral shedding (FIG. 2C) were quantified by PRRSV-specific RT-qPCR in serum and nasal swabs, respectively (genomic copy numbers/mL [log 10]). The black bars represent the median values; in addition, individual data points are shown for MOCK vaccinated animals (open diamonds) and MLV vaccinated animals (filled squares). The data were analyzed using Sidak multiple comparison test 2-way ANOVA. Each vaccinated group was compared to their respective PRRSV type-2 challenge MOCK vaccinated group within each timepoint. ****p<0.0001. ***p<0.001, **p<0.01, *p<0.05.

There were no relevant differences in weight gains between the groups. Clinical signs were mild; they included lethargy and respiratory distress at around 7-14 dpc. Only two differences between MOCK- and Prevacent®-vaccinated groups were noticed: i) within NC174-challenged groups, the Prevacent®-vaccinated pigs were slightly less lethargic; and ii) in contrast to the Prevacent®-vaccinated pigs, the MOCK group exhibited pronounced clinical signs upon challenge with NADC20: MOCK-pigs showed signs of respiratory distress, strong lethargy, and anorexia from 7-14 dpc (data not shown, d.n.s.). Rectal temperatures varied between groups (FIG. 2A): only the NC174 and NADC20 challenged pigs exhibited increased temperatures at 7 dpc. Within NC174-challenged groups, the Prevacent® vaccinated pigs had even a higher temperature compared to their MOCK-vaccinated pigs. Body temperatures upon challenge with NADC20 were similar between the MOCK- and Prevacent®—vaccinated pigs. Taken together, under the present research conditions, clinical signs were mostly mild and included lethargy, anorexia, respiratory disease, and some elevated temperatures for NC174 and NADC20. The most prominent protective aspect of Prevacent® were the reduced respiratory disease, anorexia and lethargy after NADC20 challenge.

With the limited clinical signs, viral load quantification in nasal swabs and sera were performed to better evaluate the heterologous vaccine efficacy (FIG. 2B, 2C). Pre-challenge viral load analysis at 0 dpc showed that all Prevacent®-vaccinated animals had similar PRRSV viral copy numbers: this confirms that Prevacent® vaccination was not only successful but also homogenous (FIG. 2B). Challenge with the different PRRSV strains induced viremia that peaked at 7 dpc. At that time, challenge with VR2332 led to a mild to moderate viremia with a median genomic copy number of 10\hat{ }6.2 in the MOCK-vaccinated groups. In contrast, NC174, NADC30, and NADC20 challenge induced strong viremias in MOCK-vaccinated pigs: median genomic copy numbers/mL were 10\hat{ }9.0, 10\hat{ }8.6, and 10\hat{ }9.2, respectively. At 14 dpc, viremia decreased by ˜1-2 logs. For VR2332 and NADC30, Prevacent® vaccination significantly reduced viremia at both time points; for NADC30, it reduced viremia at 14 dpc (FIG. 2B). Of note, while all MOCK-vaccinated pigs remained viremic upon VR2332 challenge, 4/6 Prevacent®-vaccinated pigs could clear this PRRSV strain by 14 dpc.

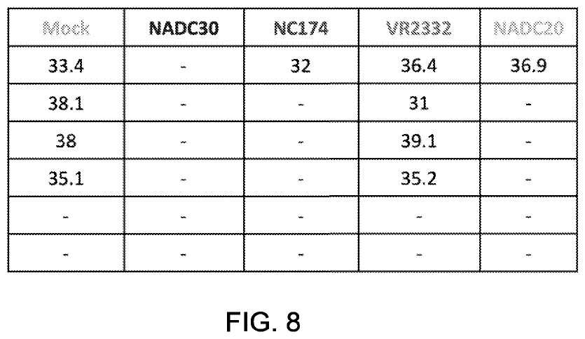

In addition to the PRRSV-generic RT-qPCR, a Prevacent®-specific RT-qPCR analysis was performed for sera at 7 and 14 dpc: this goal of this analysis was to provide insight into the contribution of the Prevacent® vaccine strain and the challenge strains to the overall PRRSV load in sera. This analysis showed that i) at 14 dpc, Prevacent® was not detected at 14 dpc (d.n.s); and ii) at 7 dpc, it was either cleared from sera or present at only very low levels (Ct≥31). Interestingly, while most animals (4/6) in the MOCK- and VR2332-challenged groups had still detectable levels of the Prevacent® vaccine strain in sera, all or 5/6 animals within the NADC30, NC174, and NADC20 groups cleared the Prevacent® vaccine strain. These data show that pigs challenged with PRRSV strains that induce high viremia, cleared the Prevacent® strain faster (FIG. 8, and d.n.s.).

FIG. 8 shows the quantification of the Prevacent® vaccine strain in serum. The prevalence of the Prevacent® vaccine strain was quantified via Prevacent®-specific RT-qPCR. This table shows the Ct values of MOCK, NADC20, NC174, VR2332, and NADC20 challenged animals at 7 days post challenge.

In addition to viremia, viral loads were also assessed in nasal swabs to evaluate viral shedding. Importantly, the Prevacent® vaccine strain was not detected in nasal swabs at the analyzed time points −0, 7, and 14 dpc (=28, 35, and 42 dpv, respectively; d.n.s.). In VR2332 challenged pigs, viral loads in nasal swabs were either low (<10\hat{ }4 genomic copy number/ml in 4/12 pigs) or completely absent (8/12 pigs; FIG. 2C). In contrast, challenge with the other PRRSV strains led to considerably higher viral loads in nasal swabs of all inoculated animals (include mean genomic copy number range; FIG. 2C). As viremia, viral loads in nasal swabs peaked at 7 dpc. Prevacent® vaccination led by number to a decrease in viral loads in nasal swabs at 7 dpc for NC174, NADC20 and NADC30. At 14 dpc, Prevacent® vaccination both significantly reduced and completely cleared the viral loads in nasal swabs of NADC20 and NADC30 challenged pigs.

FIG. 3A-C depicts the heterologous vaccine efficacy on lung viral loads, pathology, and inguinal lymph nodes size. (FIG. 3A) Viral loads in lung were assessed in bronchoalveolar lavage (BAL) by PRRSV-specific RT-qPCR (genomic copy numbers/mL [log 10]). Of note, while PRRSV was not detected in BAL of one MOCK-vaccinated/NADC20-challenged animal, this pig had the second highest lung pathology score combined with the highest PRRSV load in the 14 dpc nasal swab. Based on this discrepancy, it was excluded from this analysis. (FIGS. 3B and C) Lung gross- and histopathology of all seven lobes were assessed by a blinded veterinarian at 14 days post challenge (dpc). (FIG. 3B) depicts the percental lung lesions for each individual pig. (FIG. 3C) shows the histopathology scores of all seven lobes following the scoring guidelines of Halbur et al. See Halbur et al. “Comparison of the pathogenicity of two US porcine reproductive and respiratory syndrome virus isolates with that of the Lelystad virus,” (1995) Vet Pathol. 32(6): pp. 648-660. Each vaccinated group was compared to their respected PRRSV type-2 challenge unvaccinated group using a two-tailed unpaired t-test. The black bars represent the median values; in addition, individual data points are shown for MOCK vaccinated animals (open diamonds) and MLV vaccinated animals (filled squares). Each vaccinated group was compared to their respective PRRSV type-2 challenge unvaccinated group. Data comparison was performed using a two-tailed unpaired t-test. ****p<0.0001. ***p<0.001, **p<0.01, *p<0.05.

At necropsy, viral loads were additionally quantified via universal PRRSV-specific qPCR in BAL (FIG. 3A). All MOCK-challenged pigs were PRRSV-2 negative in BAL. MOCK-vaccinated pigs showed with ˜10\hat{ }8 genomic copy numbers per mL BAL the highest median viral loads in all challenge groups. Prevacent® vaccination could drop these viral loads by number to 10\hat{ }6.5 for NC174, and significantly to 10\hat{ }3.7 for NADC30, 10\hat{ }5.3 for NADC20, and even eliminated VR2332 in the BAL from 4/6 pigs. In conclusion, Prevacent® was absent in nasal swabs at 4-6 weeks post vaccination; and it was either absent or present at low levels in sera at these time points.

Prevacent® vaccination significantly reduced viremia upon VR2332 challenge; it limited both viremia and viral shedding in NADC20 and NADC30 challenged groups; and Prevacent® significantly reduced the BAL viral loads of NADC20, NADC30, and VR2332 (FIG. 3A).

### Vaccine Efficacy in Tissues—Viral Loads, Lung Pathology, and Lymph Node Sizes

At necropsy, viral loads were assessed via PRRSV-specific qPCR in BAL (FIG. 3A). All MOCK-challenged animals were negative. MOCK-vaccinated animals from the NADC30, NC174, and NADC20 groups had viral loads of ˜10\hat{ }8 genomic copy numbers/mL. In contrast, the MOCK-inoculated and VR2332 challenged animals had with ˜10\hat{ }5 a roughly 1,000× fold lower median viral load. Prevacent® vaccination could reduce by number the median viral loads of all challenge strains. This reduction became significant for NADC30, VR2332, and NADC20. Of note, the BAL from 4/6 Prevacent® vaccinated and VR2332 challenged animals were PRRSV-2 negative.

Lung gross pathological changes were mainly absent in both MOCK challenged groups and minimal in VR2332 challenged groups; yet, they were clearly present in NC174, NADC20, and NADC30 challenged pigs (FIG. 3B). Prevacent® vaccination reduced the lung gross pathology in challenged with two of the three pathology-inducing strains—NADC30 and NADC20. As seen in gross pathology, median histopathological changes in MOCK vaccinated pigs were also highest in the NADC30, NC174, and NADC20 groups.

However, histopathological changes were also present in the MOCK and VR2332 challenged groups (FIG. 3C). Comparing MOCK and Prevacent® vaccinated groups, the histopathology analysis revealed only one difference: Prevacent®-vaccinated animals had significantly lower histopathology scores compared to MOCK-vaccinated animals.

In addition to lung pathology, lymph nodes were assessed for size as an increase in lymph node size is often associated with inflammation. Both MOCK and VR2332 groups remained around about a healthy weight at necropsy (median weigh ˜2 grams). PRRSV-2 challenge caused the lymph nodes to enlarge for NC174, NADC20 and, NADC30. Prevacent® was able to significantly reduce the lymph node size for NADC30 by one gram (4.7 grams to 3.6 grams median weight).

Conclusively, Prevacent® reduced the BAL viral loads for NADC30, VR2332, and NADC20. While VR2332 only induced minimal lung pathology, Prevacent® did reduce not only lung gross- and/or histopathology but also the median inguinal lymph node weights for NADC30 and (by number) for NADC20.

### Examples 2-6

### Heterologous Vaccine Immunogenicity and Immune Correlates of Protection (CoP)

### SUMMARY

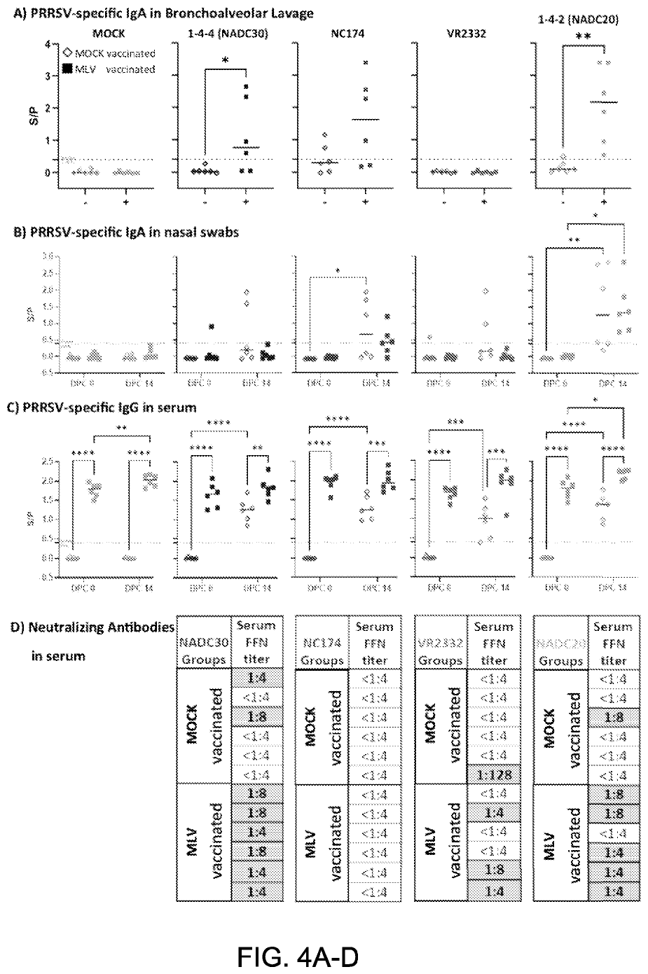

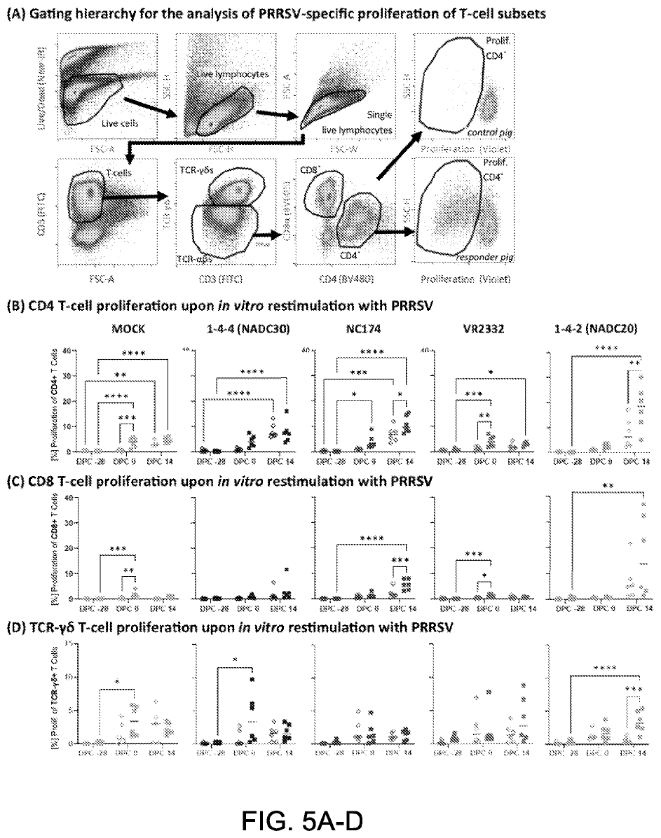

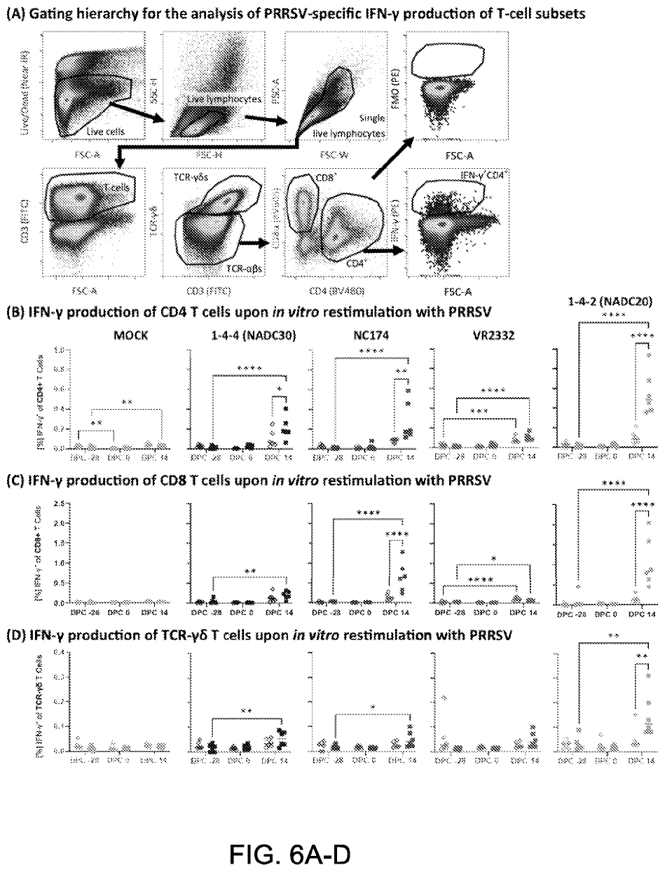

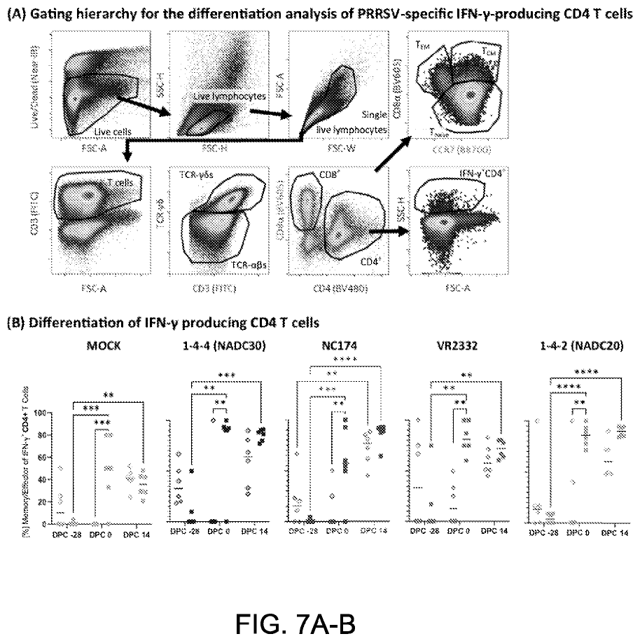

In addition to vaccine efficacy, heterologous vaccine immunogenicity was investigated for both the humoral and T-cell immune response in order to determine immune correlates of protection (CoP). The humoral immune response was studied by quantifying the local anti-PRRSV IgA levels in nasal swabs and BAL, and the serum anti-PRRSV IgG and nAb levels (FIG. 4A-D). To study the T-cell response, PBMC were isolated, in vitro restimulated with the respective PRRSV challenge strains, and analyzed via polychromatic flow cytometry for three main readout parameters—i) proliferation (FIG. 5A-D) and ii) IFN-γ production (FIG. 6A-D) of CD4, CD8, and TCR-γδ T cells, and iii) CD4 T-cell differentiation into memory/effector cells (FIG. 7A-B).

### Example 2

### Humoral Immune Response

The local humoral immune response was studied by quantifying anti-PRRSV IgA levels in BAL at necropsy (FIG. 4A) and in nasal swabs at 0 and 14 dpc (FIG. 4B).

FIG. 4A-D depicts the heterologous vaccine immunogenicity as the humoral immune response. Immunoglobulin A of (FIG. 4A) bronchoalveolar lavage (BAL), (FIG. 4B) nasal swabs, and (FIG. 4C) serum IgG levels were evaluated at 0 and 14 days post challenge (dpc) via a PRRSV×3 ELISA. The IgA and IgG ELISA S/P ratios were compared within their challenge groups—MOCK (grey), 1-4-4 (NADC30, dark blue), NC174 (red), VR2332 (green), and 1-4-2 (NADC20, light blue). The black bars represent the median values; in addition, individual data points are shown for MOCK vaccinated animals (open diamonds) and MLV vaccinated animals (filled squares). Data were statistically analyzed using a 2-way ANOVA with time and vaccination as the two parameters and Tukey's multiple comparison test. ****p<0.0001. ***p<0.001, **p<0.01, *p<0.05. (FIG. 4D) Neutralizing antibody (NA) titers determined via FFN test against the respective challenge strain at 0 and 14 dpc. Since no animals showed FFN titers at 0 dpc, only the 14 dpc data are shown. Titers≥1:4 were considered positive. The titer for each individual PRRSV-challenged pig is shown. Positive NA titers are highlighted in blue (NADC30), red (NC174, not detected), green (VR2332), and light blue (NADC20).

Of note, in contrast to the non-diluted nasal swab samples, the BAL samples were diluted 1:200 before analysis. At two weeks post challenge, the majority of BAL samples from MOCK-vaccinated pigs were negative for PRRSV-specific IgA. While the BAL from vaccinated and VR2332 challenged pigs were also negative, Prevacent® vaccination induced a strong IgA response for NADC30, NC174, and NADC20: 4/6 of the Prevacent®-vaccinated pigs in the NADC30 and NC174 pigs and all Prevacent® vaccinated pigs in the NADC20 challenge group had S/P ratios of >0.4. Thereby, Prevacent increased the lung PRRSV-specific IgA levels for NC174 (by number) and significantly for NADC30 and NADC20.

The IgA levels in nasal swabs were considerably lower (FIG. 3B): except for some outliers, IgA levels in the MOCK, NADC30 and VR2332 challenged pigs remained below an S/P ratio of 0.4. However, challenge with NC174 and NADC20 induced an observable and mostly significant local IgA response. Yet, there was no difference between the respective MOCK and Prevacent® vaccinated groups.

The systemic humoral immune response was evaluated in two ways—anti-PRRSV IgG and challenge-strain specific nAb levels in serum (FIG. 4C, 4D). Four weeks post vaccination, so at 0 dpc, every vaccinated animal but no control animal had a high positive IgG level—S/P 1.3-2.1. By 14 dpc, infection with each of the four PRRSV strains also induced anti-PRRSV serum IgG in MOCK-vaccinated animals; yet, all vaccinated animals had significantly higher serum IgG levels than their respective MOCK-vaccinated groups (FIG. 4C).

The challenge-strain specific serum nAb titers were determined by an FFN test at 0 and 14 dpc (FIG. 4D). No nAbs were detected at 0 dpc (d.n.s). At 14 dpc, neither the MOCK (d.n.s.) nor the NC174 challenged groups developed nAbs against the challenge strain either. However, NADC20, NADC30 and VR2332 challenge induced mainly low-titer serum nAbs by 14 dpc: out of the six pigs per group, only 1-2 pigs developed serum nAb titers in the MOCK-vaccinated animals; in contrast, 3/6, 5/6 and 6/6 pigs in the Prevacent®-vaccinated groups developed nAb against the VR2332, NADC20, and NADC30 challenge strains, respectively (FIG. 4D).

These data demonstrate that Prevacent® vaccination induced a strong local IgA response in BAL for NADC30, NC174 (by number), and NADC20; it also induced a systemic humoral immune response with high serum IgG titers in all groups and a higher post-challenge frequency of nAb positive animals against VR2332, NADC30 and NADC20.

### Example 3

### Proliferation of T-Cell Subsets

In addition to the humoral response, the cellular immune response is crucial for the protection against PRRSV and can provide a model for evaluating immune correlates of protection (CoP). To provide a more detailed understanding of the MLV-induced heterologous vaccine immunogenicity, the PRRSV-strain specific proliferative (FIG. 5A-D) and IFN-γ (FIG. 6A-D) response of CD4, CD8, and TCR-γδ T cells was investigated.

FIG. 5A-D shows the heterologous vaccine immunogenicity as the proliferation of CD4, CD8, and TCR-γδ T cells. (FIG. 5A) shows the gating hierarchy to assess the heterologous proliferative response of T-cell subsets to the respective PRRSV type-2 challenge strains. A live/dead discrimination dye was included to exclude dead cells. Live cells were used to identify live lymphocytes via a FSC/SSC lymphocyte gate. From live lymphocytes, doublets were excluded using a FSC-width (FSC-W)/FSC-area (FSC-A) gate on singlets. These single living lymphocytes were used to gate on T cells (FSC-A/CD3), and further to discriminate TCR-αβ and TCR-γδ T cells. TCR-αβ T cells were further divided into CD4 and CD8 T cells via their CD4/CD8α expression profile. Proliferation of the CD4, CD8, and TCR-γδ T cells was identified via a violet proliferation dye. Two examples demonstrate representative staining patterns of a control animal (top right plot) and a high responder animal (bottom right plot). (FIG. 5B-D) show the proliferative responses of CD4 (FIG. 5B), CD8 (FIG. 5C), and TCR-γδ T cells (FIG. 5D) according to their challenge groups—MOCK (grey), NC174 (red), NADC20 (light blue), NADC30 (dark blue) and VR2332 (green). The black bars represent the median values; in addition, individual data points are shown for MOCK vaccinated animals (open diamonds) and MLV vaccinated animals (filled squares). Data were statistically analyzed using a 2-way ANOVA with time and vaccination as the two parameters and Tukey's multiple comparison test. ****p<0.0001. ***p<0.001, **p<0.01, *p<0.05.

The proliferative response of CD4, CD8, and TCR-γδ T cells was analyzed after in vitro restimulation with the respective challenge PRRSV-2 strains (MOI 0.1) by multi-color flow cytometry-gating hierarchy shown in FIG. 5A. At −28 dpc, CD4 T cells showed a very limited background proliferation (FIG. 5B). Four weeks later (0 dpc) and in contrast to MOCK vaccinated pigs, CD4 T cells from Prevacent®-vaccinated pigs started to develop a heterologous proliferative response. This response was moderate for NADC20, clearly visible for NADC30 and NC174, and significant for the MOCK- and VR2332-challenged groups. At 14 dpc, the proliferative CD4 T-cell response was significantly increased also in Prevacent®-vaccinated animals of the NC174 and NADC20 groups. In contrast to CD4 T cells, CD8 T cells showed mostly a lower proliferative response (FIG. 5C). Yet, they displayed a similar pattern: i) generally, the proliferative response was increasing over time; ii) at 0 dpc, mainly the MOCK and VR2332 groups experienced an increased proliferation; and iii) post-challenge, Prevacent® could boost their proliferation in the NC174 group. The proliferative TCR-γδ response showed a higher within-group variability. Comparing MOCK- and Prevacent® groups, the only clear and significant effect of Prevacent was an increase in TCR-γδ at 14 dpc against the NADC20 strain (FIG. 5D).

Collectively, these data indicate that while the effect on TCR-γδ proliferation was limited to NADC20, Prevacent® vaccination increased the proliferation of CD4 and CD8 T cells against three heterologous PRRSV-2 strains—VR2332 (pre-challenge), NC174 (post-challenge), and NADC20 (CD4 T cells only, post-challenge).

### Example 4

### IFN-γ Production of T-Cell Subsets

In addition to the systemic proliferative response, heterologous vaccine immunogenicity was also evaluated by studying the arguably most relevant antiviral T-cell cytokine—IFN-γ (FIG. 6A-D).

FIG. 6A-D show the heterologous vaccine immunogenicity as IFN-γ production of CD4, CD8, and TCR-γδ T cells. (FIG. 6A) Gating hierarchy to assess the heterologous IFN-γ response of T-cell subsets to the respective PRRSV type-2 challenge strains. The gating hierarchy follows largely the proliferation analysis shown in FIG. 5. However, instead of gating on proliferating cells, IFN-γ was analyzed in a FSC-A/IFN-γ plot. The IFN-γ gate was set using the appropriate FMO control (top right plot). (FIG. 6B-D) show the IFN-γ responses of CD4 (FIG. 6B), CD8 (FIG. 6C) and TCR-γδ T cells (FIG. 6D) according to their challenge groups—MOCK (grey), 1-4-4 (NADC30, dark blue), NC174 (red), VR2332 (green), and 1-4-2 (NADC20, light blue). The black bars represent the median values; in addition, individual data points are shown for MOCK vaccinated animals (open diamonds) and MLV vaccinated animals (filled squares). Data were statistically analyzed using a 2-way ANOVA with time and vaccination as the two parameters and Tukey's multiple comparison test. ****p<0.0001. ***p<0.001, **p<0.01, *p<0.05.

The gating hierarchy used to selectively analyze the IFN-γ production of CD4, CD8, and TCR-γδ T cells is similar to the proliferation analysis and shown in FIG. 6A. Pre-challenge (−28 and 0 dpc), IFN-γ production was low in all T-cell subsets with no significant differences between the respective vaccination groups (FIG. 6B-D). In contrast, at 14 dpc, there was a notable IFN-γ production in most PRRSV-2 challenged groups. For CD4 T cells, this post-challenge response was significantly increased by Prevacent® vaccination against NC174, NADC20, and NADC30 (FIG. 6B). In CD8 T cells. Prevacent® significantly boosted the IFN-γ response against NC174 and NADC20 (FIG. 6C); and for TCR-γδ T cells, Prevacent® vaccination led to an increased IFN-γ production against NADC20 (FIG. 6D). Therefore, Prevacent® vaccination could boost the heterologous post-challenge IFN-γ response against NADC30 (in CD4 T cells), NC174 (in CD4 and CD8 T cells), and NADC20 (in all T-cell subsets).

### Example 5

### Differentiation of IFN-γ Producing CD4 T Cells

While the heterologous IFN-γ production showed the strongest response post-challenge, T-cell differentiation analysis of the IFN-γ producing T cells revealed remarkable pre-challenge differences (FIG. 7A-B).

FIG. 7A-B depict the heterologous vaccine immunogenicity as the differentiation of IFN-γ producing CD4 T cells. (FIG. 7A) shows the gating hierarchy to assess the differentiation of IFN-γ producing CD4 T cells. After gating on IFN-γ+CD4 T cells as described in FIG. 6, their differentiation was analyzed via their CD4/CD8a expression profile to distinguish naïve (CCR7+CD8α−), central memory (T_{CM} CCR7+CD8α+) and effector memory (T_{EM}, CCR7-CD8α+) CD4 T cells (top right plot). Since the vast majority of CD8α+IFN-γ-producing CD4 T cells belonged to the T_{CM} subset (data not shown), both T_{CM} and T_{EM} were combined in the downstream analysis into the “memory/effector” subset. (FIG. 7B) shows the frequency of these memory/effector within IFN-γ-producing CD4 T cells according to their challenge groups—MOCK (grey), 1-4-4 (NADC30, dark blue), NC174 (red), VR2332 (green), and 1-4-2 (NADC20, light blue). The black bars represent the median values; in addition, individual data points are shown for MOCK vaccinated animals (open diamonds) and MLV vaccinated animals (filled squares). Data were statistically analyzed using a 2-way ANOVA with time and vaccination as the two parameters and Tukey's multiple comparison test. ****p<0.0001. ***p<0.001, **p<0.01, *p<0.05.

Once more, a multi-color flow cytometry with a sophisticated gating hierarchy was used to assess the differentiation of IFN-γ producing CD4 T cells into CCR7⁺CD8α⁻ naïve, CCR7⁺CD8α⁺ central memory (T_{CM}) and CCR7⁻, CD8α⁺ effector memory (T_{EM}) CD4 T cells (FIG. 7A). Since the vast majority of IFN-γ producing CD8α⁺ CD4 T cells belonged to the CCR7⁺ T_{CM} subset (data not shown), the antigen-experienced T_{CM} and T_{EM} subsets were combined into one “memory/effector” subset (FIG. 7B). Pre-vaccination, so at −28 dpc, the majority of IFN-γ was produced by naïve CD4 T cells—median: 10-40% memory/effector CD4 T cells. At 0 dpc, IFN-γ in the MOCK-vaccinated groups was still mainly produced by naïve CD4 T cells—median 0-15% memory/effector CD4 T cells.

In contrast, in Prevacent®-vaccinated groups, IFN-γ was mainly produced by memory/effector CD4 T cells—median ˜50% to >90%. This difference in pre-challenge differentiation of IFN-γ producing CD4 T cells was significant for all groups and PRRSV-2 challenge strains. At 14 dpc, the frequency of memory/effector CD4 T cells increased in the MOCK-vaccinated groups; yet, at least by number, the Prevacent®-vaccinated groups still had a higher median frequency of memory/effector cells within all PRRSV-2 challenged groups (FIG. 7B). Conclusively, this CD4 differentiation analysis reveals an important immune mechanism in heterologous vaccine immunogenicity: while Prevacent® increased the CD4 IFN-γ response not before challenge, it already promoted the pre-challenge differentiation of these CD4 T cells against every analyzed PRRSV-2 strain—NC174, NADC20, NADC30, and VR2332.

### Example 6

### Immune Correlates of Protection (CoP)

The data above demonstrate that Prevacent® showed various degrees of heterologous vaccine immunogenicity and efficacy. An important parameter barely analyzed for PRRSV are immune correlates of protection (CoP). See Plotkin et al., “Nomenclature for immune correlates of protection after vaccination,” (2012) Clin Infect Dis 54(11): pp. 1615-1617. These correlates can facilitate vaccine development as well as forecasting of vaccine efficacy against emerging PRRSV strains.

To provide insight into potential CoPs for heterologous PRRSV strains, the inventors performed correlation analysis between the analyzed pre-challenge immune parameters (0 dpc) and three post-challenge (14 dpc) parameters associated with protection—lung pathology, viral shedding, and viremia (Table 2).

TABLE 2
(A), (B), (C): Immune correlates of protection.
A)

| Protection | | Gross pathology | | | |
|---|---|---|---|---|---|
| Response (0 dpt) | | NADC30 | NC174 | VR2332 | NADC20 |
| TCR-γδs | Proliferation | −0.37 | −0.27 | 0.01 | −0.42 |
| | IFN-γ | −0.23 | 0.28 | −0.26 | 0.46 |
| Cd8 | Proliferation | −0.37 | 0.34 | −0.22 | −0.09 |
| | IFN-γ | 0.26 | 0.54 | 046 | 0.11 |
| Cd4 | Proliferation | −0.46 | 0.22 | −0.36 | −0.48 |
| | IFN-γ | −0.36 | −0.19 | −0.23 | −0.59 |
| | Memory | 0.02 | 0.29 | −0.13 | −0.77 |
| Humoral | Local IgA | −0.28 | 0.24 | −0.21 | −0.45 |
| | Systemic | −0.55 | 0.23 | −0.37 | −0.59 |
| | IgG | | | | |

B)

| Protection | | Shedding | | | |
|---|---|---|---|---|---|
| Response (0 dpt) | | NADC30 | NC174 | VR2332 | NADC20 |
| TCR-γδs | Proliferation | −0.50 | 0.02 | n.d | −0.19 |
| | IFN-γ | −0.19 | 0.09 | | 0.72 |
| Cd8 | Proliferation | −0.61 | 0.20 | n.d | −0.35 |
| | IFN-γ | 0.27 | −0.18 | | 0.01 |
| Cd4 | Proliferation | −0.72 | −0.40 | n.d | −0.56 |
| | IFN-γ | −0.75 | −0.43 | | −0.49 |
| | Memory | −0.61 | −0.16 | | −0.40 |
| Humoral | Local IgA | −0.35 | −0.01 | n.d | −0.54 |
| | Systemic | −0.95 | −0.18 | | −0.55 |
| | IgG | | | | |

C)

| Protection | | Viremia | | | |
|---|---|---|---|---|---|
| Response (0 dpt) | | NADC30 | NC174 | VR2332 | NADC20 |
| TCR-γδs | Proliferation | −0.42 | 0.29 | −0.17 | −0.49 |
| | IFN-γ | 0.04 | −0.11 | 0.39 | 0.36 |
| Cd8 | Proliferation | −0.45 | 0.11 | −0.41 | −0.31 |
| | IFN-γ | −0.04 | −0.23 | −0.22 | 0.21 |
| Cd4 | Proliferation | −0.64 | −0.22 | −0.57 | −0.73 |
| | IFN-γ | −0.58 | −0.17 | −0.34 | −0.52 |
| | Memory | −0.70 | −0.36 | −0.59 | −0.74 |
| Humoral | Local IgA | −0.15 | −0.36 | 0.17 | −0.60 |
| | Systemic | −0.73 | −0.27 | −0.86 | −0.78 |
| | IgG | | | | |

Table 2 lists the R values for the correlations (−1 to +1) between various immune parameters at 0 dpc (e.g., proliferation, IFN-γ production, and differentiation into CD4 memory cells) and the three protective measures (gross pathology (A), shedding (B), and viremia(C)) at 14 dpc. While numbers in italics represent non-significant correlations, the bold numbers emphasize significant correlations (p<0.05). Negative correlations (R=0>−1) indicate that an increase in the pre-challenge immune parameter correlates with a reduced pathology or viral load. Neither the systemic CD8 and TCR-γδ response correlated well with protection: only CD8 proliferation negatively correlated with NADC30 shedding; the TCR-γδ IFN-γ response even significantly correlated positive with NADC20 shedding.

In contrast, with the exception of NC174 gross pathology, the CD4 T-cell response correlated negatively with all analyzed parameters of protection. The strongest and most significant CD4 correlations were observed for the NADC20 and/or NADC30 strains: the CD4 IFN-γ response and differentiation into memory/effector cells significantly correlated negative with NADC20-induced lung gross pathology; CD4 T-cell proliferation showed a significant negative correlation with NADC20 shedding and viremia; and all CD4 parameters (proliferation, IFN-γ, and differentiation) correlated with both NADC30 shedding and viremia. Regarding the humoral immune response, while IgA levels in nasal swabs (local IgA) showed both positive and negative correlations with protection, the systemic IgG levels correlated well with most protection parameters: systemic IgG level significantly correlated negative with NADC20-induced gross pathology, NADC30-induced shedding, and with viremia induced by NADC30, VR2332, and NADC20. These data demonstrate the while only the T-cell response was analyzed in a strain-dependent manner, both systemic IgG levels and the CD4 T-cell response are candidates to serve as important CoP for PRRSV.

### CONCLUSIONS

This current disclosure combined Prevacent® vaccination followed by in vivo challenge with four heterologous PRRSV strains with an extensive ex vivo and in vitro analysis of lung pathology, viral loads in various tissues, and the humoral and adaptive immune response. The in-depth analysis of the heterologous humoral and T-cell immune response nicely explains the immunogenicity of Prevacent®: early on (at 0 dpc), Prevacent® induces early T-cell activation and differentiation shown by an increased proliferative response of CD8 but mainly CD4 T cells. In addition, PrevacenW induced the differentiation of heterologous CD4 T cells into memory/effector cells. Downstream, this early T-cell activation and differentiation leads not only to B-cell help that drives serum IgG levels (at 0 and 14 dpc) and the frequency of nAb positive animals (14 dpc), but it also primes the vaccinated pigs for an increased post-challenge IFN-γ production (14 dpc). This induction of a combined T-cell and humoral immune response induced at least partial protection against at least three of four PRRSV strains—NADC30 (lineage 1), VR2332 (lineage 5), and NADC20 (lineage 8). The included CoP analysis revealed serum IgG levels and the CD4 T-cell response (proliferation, differentiation, and IFN-γ production) to be the best systemic CoP; however, only the CD4 T-cell response can reliably be used as CoP against specific PRRSV strains.

As will be appreciated from the descriptions herein, a wide variety of aspects and embodiments are contemplated by the present disclosure, examples of which include, without limitation, the aspects and embodiments listed below:

Method for eliciting heterologous immunogenicity against heterologous porcine reproductive and respiratory syndrome virus (PRRSV) strains to allow assessment of innate immunity and adaptive immunity following vaccinating a pig of an effective amount of a modified live PRRSV vaccine then challenging the pig with an intranasal inoculation of an amount of a live PRRSV of a known strain at least 28 days post-vaccine administration. Various measurements are obtained in the pig including temperature, and weight immediately prior to administration of the modified live PRRSV vaccine, immediately prior to the challenge with the intranasal inoculation of the known PRRSV strain, and at least 7- and 14-days post challenge. In addition, blood samples are obtained from the pig immediately prior to administration of the modified live PRRSV vaccine, immediately prior to the challenge with the intranasal inoculation of the known PRSSV strain, and at least 7- and 14-days post challenge. Measured in each blood sample are, a CD4 T-cell response, the presence of strain-specific neutralizing antibodies, the presence of CD4, CD8, and TCR-γδ cells, IFN-γ levels, and the amount of PRRSV-specific immunoglobulin A (IgA) and immunoglobulin G (IgG) levels.

In this method, the administering to a pig of an effective amount of a modified live PRRSV vaccine followed by the challenging the pig with an intranasal inoculation of an amount of a known live PRRSV strain at least 28 days post-vaccine administration induces: i) an increase in T-cell activation as evidenced by a differentiation of CD4 T and CD8 cells; ii) an increase in the amount of PRRSV-specific immunoglobulin G (IgG) levels; iii) the production of serum neutralizing antibodies; and, iv) an increase of serum IFN-γ levels.

This method further comprising the isolation, storage and banking of peripheral blood mononuclear cells (PBMC) obtained from the blood samples from pigs administered an effective amount of a modified live PRRSV vaccine.

Another method disclosed is a method for determining the efficacy of a vaccine against porcine reproductive and respiratory syndrome virus (PRRSV) comprising: i) administering to a pig of an effective amount of a modified live PRRSV vaccine; ii) challenging the pig with an intranasal inoculation of an amount of a live PRSSV known strain at least 28 days post-vaccine administration; iii) measuring in the pig, temperature, and weight immediately prior to administration of the modified live PRRSV vaccine, immediately prior to the challenge with the intranasal inoculation of the known PRRSV strain, and at least 7- and 14-days post challenge; iv) obtaining blood samples, nasal swabs from the pig immediately prior to administration of the modified live PRRSV vaccine, immediately prior to the challenge with the intranasal inoculation of the known PRRSV strain, and at least 7- and 14-days post challenge; v) assessing lung and lymph node pathology in the pig upon necropsy; vi) obtaining a bronchoaveolar lavage samples upon necropsy; vii) measuring in each blood sample, nasal swab, and bronchoaveolar lavage sample, the amount of virus present, the amount of PRRSV-specific immunoglobulin A and immunoglobulin G.

In this method, where the administering to a pig of an effective amount of a modified live PRRSV vaccine, the challenging the pig with an intranasal inoculation of an amount of a known live PRRSV strain at least 28 days post-vaccine administration induces: i) little or no lung and lymph node pathology in the pig upon necropsy; ii) a decrease in the amount of PRRSV virus in samples obtained from blood, nasal swabs and bronchoaveolar lavage upon necropsy; and, iii) an increase in the amount of PRRSV-specific immunoglobulin A and immunoglobulin G.

In still another method disclosed is a method of predicting the efficacy of a vaccine against porcine reproductive and respiratory syndrome virus (PRRSV) a pig suspected of having an infection with PRRSV, comprising: i) isolating PRRSV from a blood or nasal swab sample obtained from the pig suspected of having an infection with PRRSV; ii) challenging with the PRRSV from the pig suspected of having an infection with PRRSV, isolated, stored and banked samples of peripheral blood mononuclear cells (PBMC) previously obtained from the blood samples from pigs administered an effective amount of a modified live PRRSV vaccine wherein the pigs were further challenged with an intranasal inoculation of an amount of a live PRSSV known strain at least 28 days post-vaccine administration and their PBMC's isolated, stored and banked wherein the CD4 T and CD8 T-cell response as the differentiation of CD4 T and CD8 cells was previously obtained, iii) measuring the CD4 T and CD8 T-cell response in the PBMC's following the challenge with the PRRSV from the pig suspected of having an infection with PRRSV; and iv) comparing the CD4 T and CD8 T-cell response of step iii) with the previously obtained CD4 T and CD8 T-cell response from the isolated, stored and banked PBMC samples of step ii).

In any of the disclosed methods, the porcine reproductive and respiratory syndrome virus (PRRSV) infection can be caused by an infection from any PRRSV strain.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for eliciting heterologous immunogenicity against heterologous porcine reproductive and respiratory syndrome virus (PRRSV) strains to allow assessment of innate immunity and adaptive immunity comprising:
i) administering to a pig of an effective amount of a modified live PRRSV vaccine or a control injection;
ii) challenging the pig with an intranasal inoculation of an amount of a live PRRSV of a known strain at least 28 days post-vaccine administration;
iii) measuring in the pig, temperature, and weight immediately prior to administration of the modified live PRRSV vaccine, immediately prior to the challenge with the intranasal inoculation of the known PRRSV strain, and at least 7- and 14-days post challenge;
iv) obtaining blood samples from the pig immediately prior to administration of the modified live PRRSV vaccine, immediately prior to the challenge with the intranasal inoculation of the known PRSSV strain, and at least 7- and 14-days post challenge;
v) measuring in each blood sample, a CD4 T-cell response, the presence of strain-specific neutralizing antibodies, the presence of CD4, CD8, and TCR-γδ cells, IFN-γ levels, and the amount of PRRSV-specific immunoglobulin A (IgA) and immunoglobulin G (IgG) levels; and,
vi) comparison of all measurements with those obtained from control injected pigs.

2. The method of claim 1, wherein, the known porcine reproductive and respiratory syndrome virus (PRRSV) strain is selected from PRRSV type-I (PRRSV-1) and PRRSV type-2 (PRRSV-2) virus strain.

3. The method of claim 2, wherein the known porcine reproductive and respiratory syndrome virus (PRRSV) strain is the PRRSV type-2 (PRRSV-2) virus strain selected from the group consisting of NADC30 and NC174 (lineage 1), VR2332 (lineage 5), and NADC20 (lineage 8).

4. The method of claim 1, wherein the strain-specific neutralizing antibodies are one or more of anti-NADC30, anti-VR2332, and anti-NADC20 neutralizing antibodies.

5. The method of claim 1, wherein the administering to a pig of an effective amount of a modified live PRRSV vaccine followed by the challenging the pig with an intranasal inoculation of an amount of a known live PRRSV strain at least 28 days post-vaccine administration induces:
i) an increase in T-cell activation as evidenced by a differentiation of CD4 T and CD8 cells;
ii) an increase in the amount of PRRSV-specific immunoglobulin G (IgG) levels;
iii) the production of serum neutralizing antibodies; and,
iv) an increase of serum IFN-γ levels,
as compared to control injections.

6. The method of claim 1, further comprising the isolation, storage and banking of peripheral blood mononuclear cells (PBMC) obtained from the blood samples from pigs administered an effective amount of a modified live PRRSV vaccine.

7. A method for determining the efficacy of a vaccine against porcine reproductive and respiratory syndrome virus (PRRSV) comprising:
i) administering to a pig of an effective amount of a modified live PRRSV vaccine;
ii) challenging the pig with an intranasal inoculation of an amount of a live PRSSV known strain at least 28 days post-vaccine administration;
iii) measuring in the pig, temperature, and weight immediately prior to administration of the modified live PRRSV vaccine, immediately prior to the challenge with the intranasal inoculation of the known PRRSV strain, and at least 7- and 14-days post challenge;
iv) obtaining blood samples, nasal swabs from the pig immediately prior to administration of the modified live PRRSV vaccine, immediately prior to the challenge with the intranasal inoculation of the known PRRSV strain, and at least 7- and 14-days post challenge;
v) assessing lung and lymph node pathology in the pig upon necropsy;
vi) obtaining a bronchoaveolar lavage samples upon necropsy;
vii) measuring in each blood sample, nasal swab, and bronchoaveolar lavage sample, the amount of virus present, the amount of PRRSV-specific immunoglobulin A and immunoglobulin G; and,
viii) comparison of all measurements with those obtained from control injected pigs.

8. The method of claim 6, wherein, the known porcine reproductive and respiratory syndrome virus (PRRSV) strain is selected from PRRSV type-1 (PRRSV-1) and PRRSV type-2 (PRRSV-2) virus strain.

9. The method of claim 7, wherein the known porcine reproductive and respiratory syndrome virus (PRRSV) is the PRRSV type-2 (PRRSV-2) virus strain selected from the group consisting of NADC30 and NC174 (lineage 1), VR2332 (lineage 5), and NADC20 (lineage 8).

10. The method of claim 6, wherein the administering to a pig of an effective amount of a modified live PRRSV vaccine followed by the challenging the pig with an intranasal inoculation of an amount of a known live PRRSV strain at least 28 days post-vaccine administration induces:
i) little or no lung and lymph node pathology in the pig upon necropsy;
ii) a decrease in the amount of PRRSV virus in samples obtained from blood, nasal swabs and bronchoaveolar lavage upon necropsy; and,
iii) an increase in the amount of PRRSV-specific immunoglobulin A and immunoglobulin G;
as compared to measurements with those obtained from control injected pigs.

11. A method of predicting the efficacy of a vaccine against porcine reproductive and respiratory syndrome virus (PRRSV) a pig suspected of having an infection with PRRSV, comprising:
i) isolating PRRSV from a blood or nasal swab sample obtained from the pig suspected of having an infection with PRRSV;
ii) challenging with the PRRSV from the pig suspected of having an infection with PRRSV, isolated, stored and banked samples of peripheral blood mononuclear cells (PBMC) previously obtained from the blood samples from pigs administered an effective amount of a modified live PRRSV vaccine wherein the pigs were further challenged with an intranasal inoculation of an amount of a live PRSSV known strain at least 28 days post-vaccine administration and their PBMC's isolated, stored and banked wherein the CD4 T and CD8 T-cell response as the differentiation of CD4 T and CD8 cells was previously obtained,
iii) measuring the CD4 T and CD8 T-cell response in the PBMC's following the challenge with the PRRSV from the pig suspected of having an infection with PRRSV; and
iv) comparing the CD4 T and CD8 T-cell response of step iii) with the previously obtained CD4 T and CD8 T-cell response from the isolated, stored and banked PBMC samples of step ii).

12. The method of claim 11, wherein, the porcine reproductive and respiratory syndrome virus (PRRSV) infection is caused by an infection from the strain selected from PRRSV type-1 (PRRSV-1) and PRRSV type-2 (PRRSV-2) virus strain.

13. The method of claim 12, wherein the porcine reproductive and respiratory syndrome virus (PRRSV) infection is caused by an infection from PRRSV type-2 (PRRSV-2) virus strain selected from the group consisting of NADC30 and NC174 (lineage 1), VR2332 (lineage 5), and NADC20 (lineage 8).
